(19) United States
(12) Patent Application Publication
(10) Pub. No.: US 2019/0070724 A1
(43) Pub. Date: Mar. 7, 2019

(54) DEVICE FOR REMOTE APPLICATION OF A SUBSTANCE

(71) Applicant: ROY LEE AMMANN, Kennewick, WA (US)

(72) Inventor: ROY LEE AMMANN, Kennewick, WA (US)

(21) Appl. No.: 15/696,587

(22) Filed: Sep. 6, 2017

(51) Int. Cl. B25J 1/04; A61M 35/00

(52) U.S. Cl. CPC B25J 1/04; A61M 35/003

(57) ABSTRACT

An Applicator (1) for remote application of a substance (1) which comprises a generally right or left closed or partially cylindrical body (100) having at least one detent (300) at the interior. The insertion of a cylindrical container of a substance into the body (100) will be held in position by force of the at least one detent (300) exerted against the inserted cylindrical container. A handle interface (400) is joined by a handle body joint (420) at the body (100) and an outwardly extending handle interface extension (430) can receive a handle which an operator grasps and extends the body (100) into position to allow the substance to be applied to a generally inaccessible location.

### FIELD OF THE INVENTION

This invention is a device used to reach a generally inaccessible location and to apply a substance at the inaccessible location.

### BACKGROUND OF THE INVENTION

The need for a person to apply a pain relieving ointment or, for example, to apply a lubricant to a generally inaccessible location in a machine, creates obstacles in personal health care and in industrial production lines. This invention enables the placement of a substance to the inaccessible location such as placement of a pain relieving medication to a person's back or a lubricant to a remote and difficult to access gear.

### SUMMARY OF THE INVENTION

The Applicator (1) for remote application of a substance (1) comprises a generally right or left closed cylindrical body (100) having at least one detent (300) at the interior where the insertion of a cylindrical container of a substance will be held in position by force of the at least one detent (300) exerted against the inserted cylindrical container. A handle interface (400) is joined by a handle body joint (420) at the body (100) and an outwardly extending handle interface extension (430) can receive a handle which an operator grasps and and extends the body (100) into position to allow the substance to be applied to a generally inaccessible location.

### BRIEF DESCRIPTION OF THE FIGURES

FIG. 1 illustrates an Applicator (1), a body (100), a body interior surface (110), a body exterior surface (120), a body top opening (130), a bottom (140), a handle interface (400), a handle Body joint (420), a handle interface extension (430) and a handle interface terminus (480).

FIG. 2 additionally shows a detent (300) and a handle interface aperture (440).

FIG. 3, in addition to FIG. 1, additionally shows handle interface connector (460).

FIG. 4. is a top plan illustrating detents (300) and the bottom (140) forming the right closed cylinder.

FIG. 5 is a section from FIG. 4 illustrating two detents (300).

FIG. 6 is a section from FIG. 2 illustrating three detents (300).

FIG. 7 is a section from FIG. 3 illustrating the handle interface connector (460) and three detents (300).

FIG. 8 is a perspective showing the bottom surface (500) forming the right closed cylinder.

FIG. 9 is a rotated section from FIG. 6 illustrating the detent extension (350) at the bottom (140) forming a partially closed right cylinder.

FIG. 10 is a rotted section from FIG. 6 illustrating a bottom extension (520) forming a partially closed right cylinder.

FIG. 11 illustrates a handle attached by handle affixing means at the handle interface (400).

FIG. 12 illustrates a perspective view showing at least one body aperture (150).

### DETAILED DESCRIPTION OF THE INVENTION

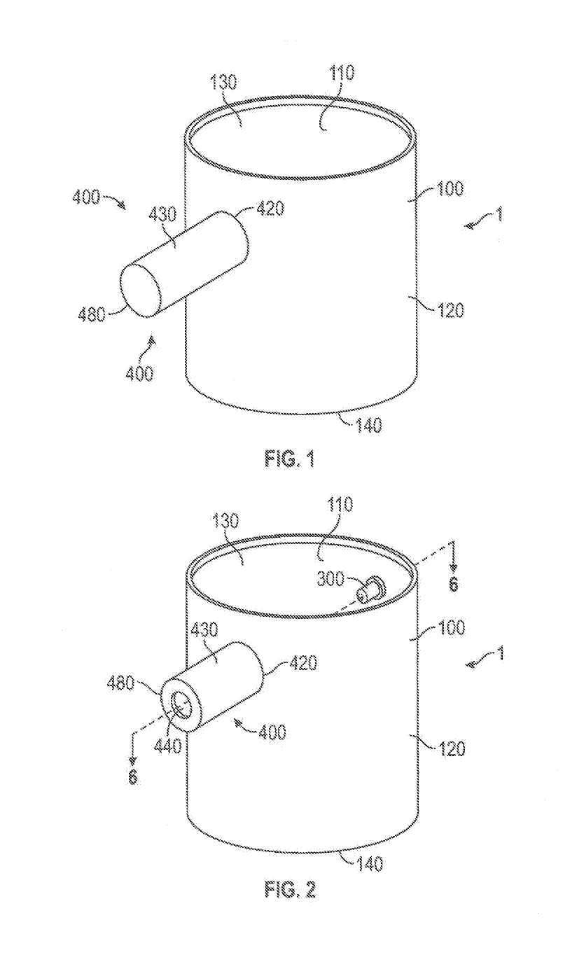

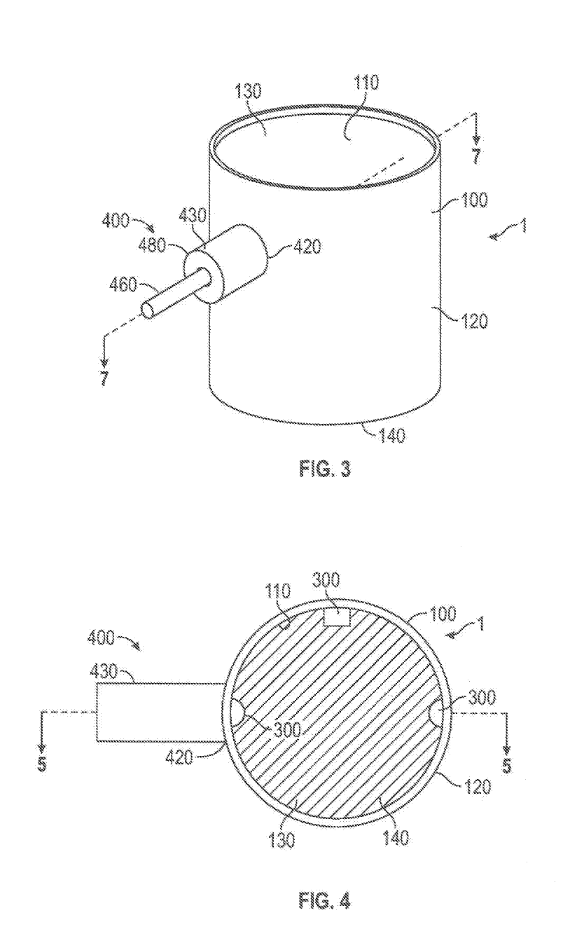

Seen in perspective view, FIGS. 1 through 3, is an Applicator (1) having a cylindrical body (100) generally circular in cross-section. The body (100) having an interior surface (110), an exterior surface (120), a top opening (130) and a bottom (140). At least one detent (300) at the interior surface (110) insures a friction fit to retain a tubular and cylindrical container when inserted into the applicator (1) at the top opening (130). In the preferred embodiment the cylindrical body (100) is right closed at the bottom (140).

An elongated handle interface (400) is rigidly fixed to the exterior surface (120) by a handle body joint (420). The applicator (1) is constructed from a rigid material, such as plastics and metals. The handle interface (400) is has an outwardly extending handle interface extension (430) which terminates at a handle interface terminus (480). When produced in a plastic injection molding process the entirety of a handle would generally be formed independently from the body (100) and will connect with the applicator (1) at the handle interface terminus (480).

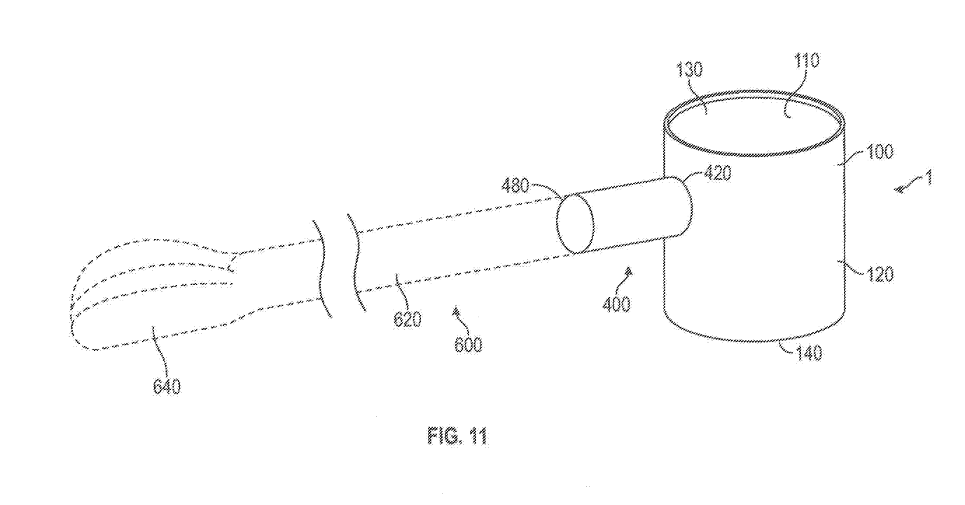

As will be appreciated by a person of ordinary skill in the arts of application to a substance to a remote location, the handle (600), shown for illustrative purposes only in FIG. 11, may have a variety of lengths and shapes. That is, in some instances the handle will have bends and or angles (620) and a handle (640) which allow the applicator (1) to apply force that premits the applicator (1) to contain a cylinder which is resistant to removal from the applicator (1), by the existence of at least one detent, and where the cylinder contains an extended substance, such as a lubricant or medication, for application to a surface at a remote location.

The handle interface (400) is the means structure to which a handle is attached or from which a handle extends. That is, a handle can be immovably affixed by mechanical devices such as clips, screws, bolt with nut, adhesives, at the handle interface terminus (480).

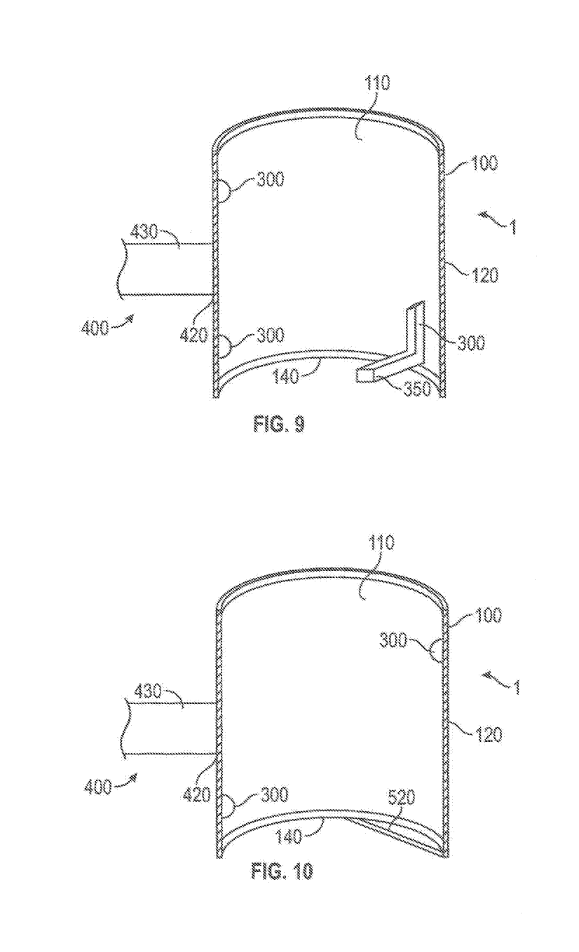

As a person of ordinary skill will recognize, there is least one detent (300) at the body interior surface (110). The person of ordinary skill will also recognize that the at least one detent may be two or more detents. And further, known by a person of ordinary skill, the at least one detent (300) may have a detent extension (350) extending outwardly from the at least one detent (300). Equally realized is that there may be a variety of shapes and configurations of the at least one detent (300), some with and some without a detent extension (350), some semihemispherical and others with irregular shapes. In an alternative embodiment, the detent extension (350) extends outwardly and impedes and stops the insertion of a cylinder thereby comprising the structure that partially closes the applicator (1) at the bottom (140) as seen in FIG. 9.

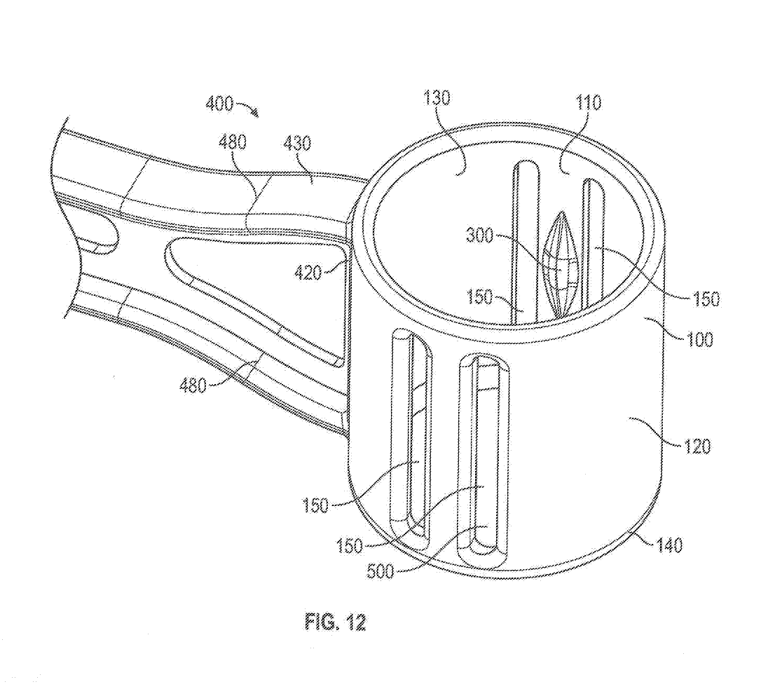

The handle interface aperture (440) is, in one embodiment, centrally positioned at the handle interface terminus (480) and extending through the handle interface extension (430) toward and proximal to the handle body joint (420). Also, as seen in FIG. 12, the body joint (420) may extend from proximal the body top opening (130) toward and proximal the bottom (140).

The interconnection between the handle interface terminus (480) may be by a handle portion being inserted into and affixed in the handle interface aperture (440) or where the handle portion having an aperture which is sized to receive an elongated and generally cylindrical handle interface connector (460) which is generally centrally positioned at the handle interface terminus (480). The handle interface connector (460) extends through the handle interface extension (430) toward and proximal to the handle body joint (420) and the handle interface connector (460) extends outwardly from the handle interface terminus (480) and outwardly from the handle body joint (420). The handle interface connector (460) is generally a rigid structural material, such as rigid plastics and metals, capable of supporting the torque which occurs when the applicator (1) is positioned to exert force against some thing at a remote location The handle interface connector (460) is elongated and is means for the attachment of a handle means.

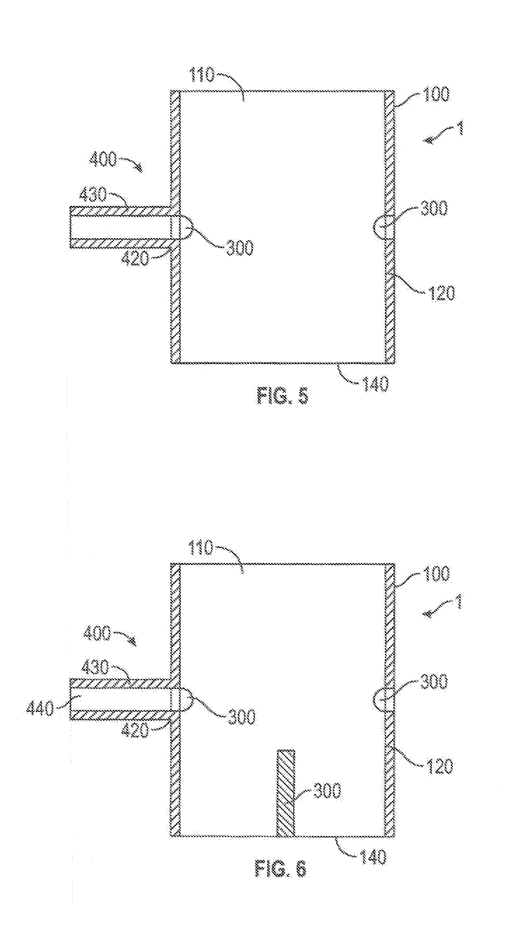

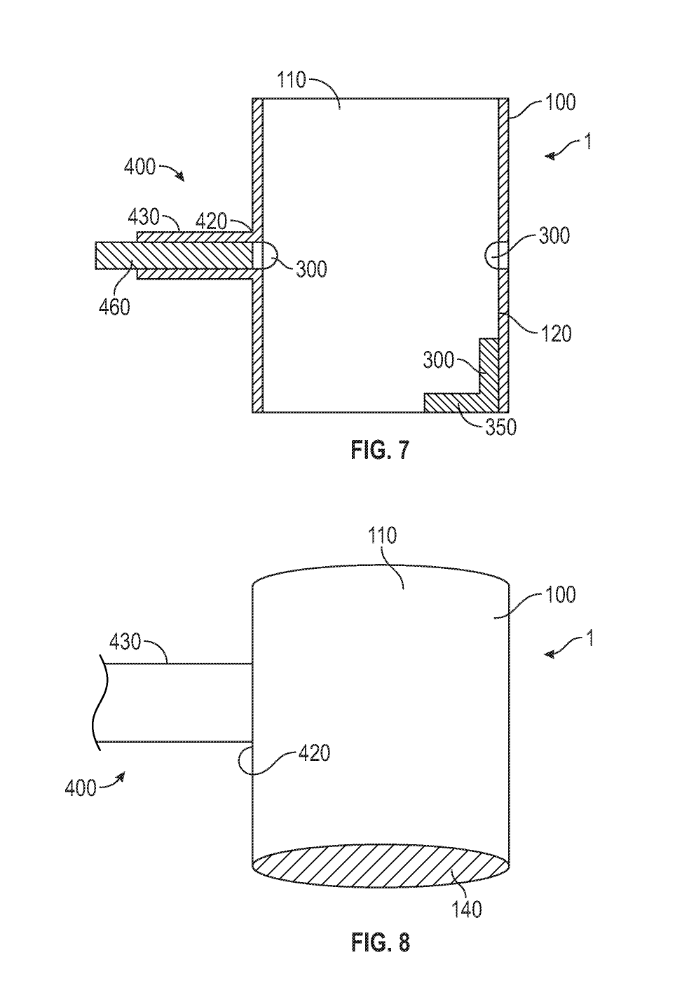

The cylindrical body (100), in the preferred embodiment, is right closed at the bottom (140) by a bottom surface (500) seen in FIGS. 1 through 12. The bottom surface (140) covering the cylindrical body (100) at the bottom (140) and closing the cylindrical body (100) at the bottom (140). However, the cylindrical body (100), in an alternative embodiment, is partially right closed at the bottom surface (500) with the bottom surface (520), seen in FIG. 10, extending outwardly from the interior surface (110) at the bottom (140) and partially closing the cylindrical body (100) at the bottom (140).

In an alternative embodiment there is at least one body aperture (150) extending from the body interior surface (110) through the body exterior surface (130). As seen in FIG. 12, the at least one body aperture (150) may be slotted extending from proximal the top opening (130) toward the bottom (140). The at least one body aperture (150) is a stress releasing device and allows some access to the inserted cylinder for removal purposes.

1. An Applicator (1) having a cylindrical body (100); the body (100) having an interior surface (110), an exterior surface (120), a top opening (130) and a bottom (140); and at least one detent (300) at the interior surface (110); the cylindrical body (100) being right closed at the bottom (140); and,
an elongated cylindrical handle interface (400) rigidly fixed to the exterior surface (120) by a handle body joint (420); the handle interface (400) having an outwardly extending handle interface extension (430) and terminating at a handle interface terminus (480); the handle interface (400) is the means structure to which a handle is attached or from which a handle extends.

2. An Applicator (1) depending from claim 1 and further comprising: at least one detent (300) having a detent extension (350) extending outwardly from the at least one detent (300).

3. An Applicator (1) depending from claim 1 and further comprising: a handle interface aperture (440) centrally positioned at the handle interface terminus (480) and extending through the handle interface extension (430) toward and proximal to the handle body joint (420).

4. An Applicator (1) depending from claim 1 and further comprising: a handle interface connector (460) centrally positioned at the handle interface terminus (480) and extending through the handle interface extension (430) toward and proximal to the handle body joint (420) and the handle interface connector (460) extending outwardly from the handle interface terminus (480) and outwardly from the handle body joint (420); the handle interface connector (460) is elongated and is means for the attachment of a handle means.

5. An Applicator (1) depending from claim 1 and further comprising: the cylindrical body (100) is right closed at the bottom (140) by a bottom surface (500); said bottom surface (140) covering the cylindrical body (100) at the bottom (140) and closing the cylindrical body (100) at the bottom (140).

6. An Applicator (1) depending from claim 5 and further comprising: the cylindrical body (100) is partially right closed at the bottom surface (500); said bottom surface (140) extending outwardly from the interior surface (110) at the bottom (140) partially closing the cylindrical body (100) at the bottom (140).

7. An Applicator (1) depending from claim 1 and further comprising: the handle interface (400) is a means structure to which a handle is attached or from which a handle extends; and
at least one body aperture (150) extending from the body interior surface (110) to the body exterior surface (130).
